(19) United States
(12) Patent Application Publication
(10) Pub. No.: US 2012/0101354 A1
(43) Pub. Date: Apr. 26, 2012

(54) SAFETY DEVICE FOR MONTORING A VASCULAR ACCESS LOCATION OF A LIVING BEING

(76) Inventor: Bernhard Ehni, Goeppingen (DE)

(21) Appl. No.: 13/282,098

(22) Filed: Oct. 26, 2011

(30) Foreign Application Priority Data
Oct. 26, 2010 (DE) 10 2010 049 723.1

(51) Int. Cl. A61B 5/026

(52) U.S. Cl. 600/371

(57) ABSTRACT

A safety device for monitoring a vascular access location on a living being includes a housing adapted to surround the vascular access location and a sensor arranged in or on the housing to detect fluid flowing from the vascular access location into the housing.

### CROSS-REFERENCE TO RELATED APPLICATION

This application claims priority of German Patent Application No. 10 2010 049 723.1, filed on Oct. 26, 2010, the subject matter of which is incorporated herein by reference.

### BACKGROUND OF THE INVENTION

The present invention relates to a safety device for monitoring a vascular access location on a living being.

For specific treatments of human patients in particular, it is necessary to infuse blood or, if applicable, other types of fluids in a controlled manner into a bloodstream or also to remove blood from a bloodstream.

One example for such a treatment is dialysis. For cleaning or purification of blood for a blood transfusion, a first cannula is inserted into an artery to remove blood from the patient so that it can be purified. A second cannula is furthermore inserted into a vein of the patient, so that the blood can again be supplied to the patient following the purification. In place of a cannula, an injection body in the form of a tube can alternatively be inserted with the aid of a catheter into the respective blood circulation.

The patient is asked to lie down on a cot for this treatment and the cannulas are then inserted into the artery and the vein. The cannulas with the attached tubes for circulating the blood are secured with tape or the like to the patient, to prevent a detaching of the cannulas.

However, securing the cannulas in this way is by no means a safe option. Even small movements of the patient can loosen and detach a cannula from the vascular access location. The danger is relatively high and should not be neglected since the dialysis treatment extends over a long period of time. A detaching of the cannula can be remedied immediately, provided nursing personnel were to remain with the patient and supervise the patient's dialysis treatment. However, since the dialysis process is very time consuming, the patient may be without supervision for long periods of time.

The patient is in greatest danger if the cannula inserted into the vein detaches itself and is pulled from the vascular access. In that case, the blood flowing back no longer reaches the patient's bloodstream, thereby resulting in extremely high blood loss within a short time. If not reached immediately by the hospital personnel, the patient in that case is in acute danger of bleeding to death.

### SUMMARY OF THE INVENTION

It is therefore an object of the present invention to provide a safety device by which such danger situations can be avoided.

The above and other objects are accomplished, according to one embodiment of the invention, by the provision a safety device for monitoring a vascular access location on a living being, comprising: a housing adapted to surround the vascular access location; and a sensor arranged in or on the housing to detect fluid flowing from the vascular access location into the housing.

The safety device according to the invention makes it possible to monitor the leakage of different types of fluids at different vascular access locations. For example, the vascular accesses lymphatic vessels can be monitored with this safety device. Without restricting the universality of the invention, the following example refers to vascular access to a bloodstream, wherein the safety device is used in particular to monitor whether a cannula or a catheter is detached from a bloodstream or, in general, to monitor the occurrence of a leakage.

The vascular access to a bloodstream can be embodied in the form of a tube which is inserted with the aid of a catheter into the bloodstream. With the safety device according to the invention, the fact is taken into account that during an unintended detaching of a cannula from the vascular access location on a patient, or in general if a leakage occurs, blood immediately flows out of the vascular access. This blood then fills the housing, wherein the housing size is advantageously dimensioned such that after a very short time interval, preferably in the range of seconds, the housing is filled at least partially with blood which can be securely detected by the sensor. The safety device then immediately generates an alarm signal, so that the patient can be helped, thereby securely preventing dangerous situations.

The safety device and the cannula with the tubes attached thereto preferably and jointly form a single functional and structural unit, so that when the cannula is inserted into the vascular access, the safety device is placed either automatically or with just a few manual interventions into the desired position. In this position, the safety device housing with the sensor rests securely and with a relatively tight fit on the skin of the patient, wherein the vascular access is arranged inside a cavity that is delimited by the housing and the skin of the patient.

In an emergency, meaning in case of an undesirable detaching of the cannula from the vascular access, the housing immediately fills up with blood which can be detected securely and within a short response time by the sensor.

The housing preferably forms together with the sensor a compact structural unit, wherein this unit occupies a small structural volume and can be produced cost-effectively. An evaluation unit for the sensor signals generated by the sensor is preferably provided as additional component for the safety device. The evaluation unit is advantageously located outside of the housing, so that its function is not hindered by blood flowing into the housing. The alarm signal is preferably also generated by the evaluation unit, wherein this signal can be an optical and/or an acoustic signal in the simplest case. Alternatively or in addition, the alarm signal can be an electric signal which is transmitted, for example, to a nursing station, so that the personnel at the nursing station can provide immediate help.

The sensor on the safety device generally operates based on the principle of a proximity switch which detects whether the housing over the vascular access is empty or filled with blood. In principle, the sensor can thus be embodied as a mechanical sensor, in particular as a piezo-electric sensor, a capacitive sensor, or an inductive sensor.

The sensor may also be an optical sensor provided with a transmitter for emitting light rays and receiver for receiving light rays. The term light in this case generally also covers electromagnetic radiation in the visible wavelength as well as in the invisible wavelength range, for example in the infrared range.

The optical sensor in this case is a light scanner which generates a binary output signal.

With a sensor embodied in this way, blood flowing into the housing can be detected securely and quickly, so that the detachment of a cannula from the vascular access can be detected reliably and after only a short response time with this optical sensor.

The optical sensor may comprise only a few components which are cheap to produce. A light-emitting diode may be used for the transmitter and a photo resistor or a photo diode may be used for the receiver.

To make possible an early detection of blood flowing from the vascular access, the transmitter and the receiver may be arranged on the inside of the housing cover so that the transmitter emits light rays in the direction of the vascular access which are reflected from there to the receiver. Alternatively, the optical sensor can also be used to realize an absorption measurement.

Provided the cannulas are inserted correctly into the vascular access, the detection sensitivity of the optical sensor can be increased further if the signals received at the receiver are stored as reference values with the aid of a teach-in process in an evaluation unit. During an operating phase that follows the teach-in process, the actually received signals are then compared to the stored reference values. If the cannulas are detached from the vascular access locations, the blood flowing out will at least partially fill the housing, thereby causing the light rays to be reflected only by the blood flowing into the housing. As a result of the deviation between the actually received signals and the reference values, the alarm signal is then generated in the evaluation unit.

The safety device according to the invention can in principle also be used in the field of veterinary medicine, but is used with particular advantage in the field of human medicine.

For dialysis treatment, the securing of a cannula inserted into a vein of a patient represents one important use of the invention. During dialysis treatment, meaning the purification of the patient's blood with the aid of a dialysis machine, blood is extracted from the patient via a cannula inserted into an artery. The blood then passes through the dialysis machine and is supplied once more to the patient via a cannula inserted into a vein. An interruption in the blood supply would result in a sudden, great loss of blood for the patient which can quickly endanger the patient's life. Danger situations of this type can be avoided with the safety device according to the invention.

The safety device according to the invention can also be used advantageously for other medical applications in which the detaching of a cannula, used to guide blood or other fluids from the vascular access of the patient, would result in considerable danger to the patient. These applications include the use in hospital intensive-care stations for inserting a catheter into a patient or for administering long-term infusions. The safety device according to the invention can be used advantageously even in cases where cannulas with attached tubes for carrying medications are attached to the patient with the aid of sewing. Finally, the device can also be used for the liquor drainage, during which liquids are suctioned from the brain or the central nervous system of a patient.

### BRIEF DESCRIPTION OF THE DRAWINGS

These and other features and advantages of the invention will be further understood from the following detailed description of embodiments of the invention with reference to the accompanying drawings.

FIG. 1 is schematic representation of the safety device according to the invention of a cannula inserted into the vascular access of a patient.

FIG. 2 is a schematic representation of the arrangement according to FIG. 1, with the cannula detached from the vascular access.

### DETAILED DESCRIPTION

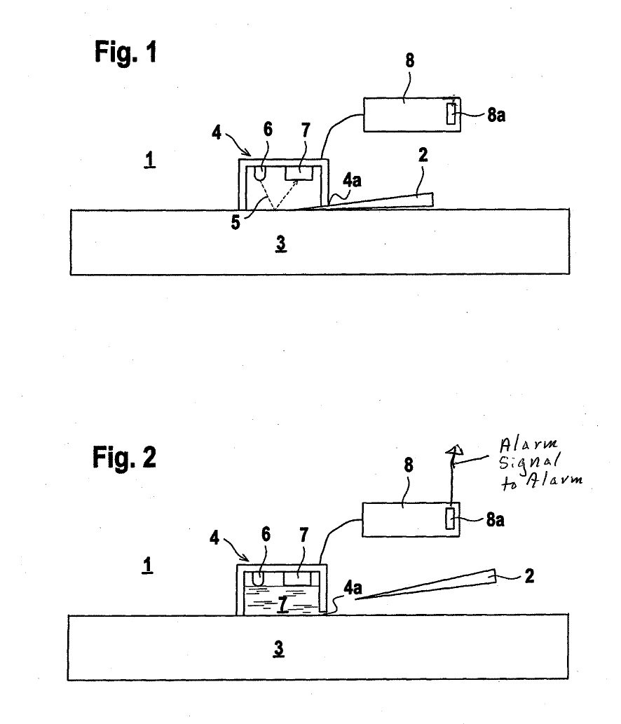

FIGS. 1 and 2 schematically illustrate an exemplary embodiment of a safety device 1 which is used to monitor whether a cannula 2, functioning as an injection body, is inserted correctly into a patient's vascular access or is detached from the vascular access. The vascular access in particular can be a puncture site, wherein reference is made to this type of application without restricting the universality of the invention. The vascular access generally can also take other forms, such as a surgical cut. FIG. 1 depicts the cannula 2 inserted at a puncture site, meaning into an arm 3 of a patient, whereas FIG. 2 shows the cannula 2 detached from the puncture site. A tube inserted via a catheter into the blood stream can generally also be used as an injection body in place of a cannula 2.

In the present case, the safety device 1 is used for monitoring the dialysis treatment of a patient. The cannula 2 in this case is connected to a tube, not shown herein, by means of which purified blood coming from a dialysis machine, not shown herein, is thus recirculated via the cannula 2 into a vein of the patient.

In the illustrated embodiment, the safety device 1 comprises a housing 4 with a therein arranged optical sensor. The housing 4 consists of an essentially cube-shaped plastic capsule which is open on the bottom. Provided in one side wall of the housing 4 can be a small opening 4a through which the cannula 2 is inserted into the housing 4 inside space. The dimensions of the housing 4 are adapted to the dimensions of the cannula 2. Thus, if the cannula 2 is inserted at the puncture site, as shown in FIG. 1, the region with the puncture site and the cannula tip is enclosed by the housing 4, once the housing is placed with the open underside onto the arm 3 of the patient.

The components of the optical sensor include a transmitter 6 for emitting light rays 5 and a receiver for receiving the light rays 5, wherein these components are arranged on the inside of the housing 4 cover. The transmitter 6 and the receiver 7 are connected to an evaluation unit 8 located outside of the housing 4. The transmitter 6 is embodied as a light-emitting diode while the receiver 7 is embodied as a photo resistor or a photo diode. The evaluation unit 8 consists of a microcontroller or the like. The optical sensor forms a light scanner for which the sensor components are dimensioned such that if the cannula 2 is correctly inserted into the puncture site, the light rays 5 emitted by the transmitter 6, or a large share thereof, are reflected back from the puncture site, meaning from the arm 3 of the patient and the cannula 2, toward the receiver 7.

An alarm is sounded if the cannula 2 shown in FIG. 2 has detached itself from the puncture site, the cavity enclosed by the arm 3 and the housing 4 quickly fills with blood, so that the light rays 5 emitted by the transmitter 6 are strongly subdued by the blood. As a result, no light rays or only a reduced number of light rays will arrive at the receiver 7. FIG. 2 illustrates the case where the housing is filled almost completely with blood.

The optical sensor distinguishes between these two cases by generating a binary output signal with two switching states. The first switching state corresponds to the error-free state where the cannula 2 is correctly inserted into the puncture site. The second switching state corresponds to an alarm signal state which indicates that blood flows from the puncture site because the cannula 2 is detached.

It makes sense to use a reference value comparison to assign the signals received by the receiver 7 to one of these switching states. In a teach-in process, the actually received signals associated with an error-free case, meaning the case where the cannula 2 is inserted correctly into the puncture site, are entered into the evaluation unit 8. These values are entered as reference values into the evaluation unit 8. During the operating phase that follows the teach-in process, the actual receiving signal values are then compared to the reference values. If the values for the actually received signals coincide with the reference values within specified tolerance limits, the evaluation unit 8 considers this an error-free state, so that the output signal assumes the first switching state. On the other hand, if the received signals are outside of the reference value tolerance range, the output signal assumes the second switching state.

The sensor output signal is emitted via an output 8a of the evaluation unit 8 and functions, for example to trigger an optical or acoustic alarm. If the first switching signal is transmitted via the output 8a, then the alarm transmitter remains deactivated. However, if the second switching state, meaning the alarm signal state, is transmitted via the output 8a, the alarm transmitter is activated and triggers an optical or an acoustic alarm, thereby alarming the hospital personnel which can then provide immediate help to the patient. The arrow in FIG. 2 represents transmission of the alarm signal from output 8a to an alarm.

It will be understood that the above description of the present invention is susceptible to various modifications, changes and adaptations, and the same are intended to be comprehended within the meaning and range of equivalents of the appended claims.

What is claimed is:

1. A safety device for monitoring a vascular access location on a living being, comprising:
a housing adapted to surround the vascular access location; and
a sensor arranged in or on the housing to detect fluid flowing from the vascular access location into the housing.

2. The safety device according to claim 1, further comprising an alarm coupled to the sensor to generate a signal when fluid leaking into the housing is detected by the sensor.

3. The safety device according to claim 1, wherein the sensor detects a detachment of a cannula or a catheter at the vascular access location.

4. The safety device according claim 1, wherein the housing has an open underside which can be fitted onto skin of the living being to create a closed housing cavity inside of which cavity the cannula or the catheter is conducted to the vascular access location.

5. The safety device according to claim 4, wherein the housing has one wall with an opening to insert the cannula or the catheter.

6. The safety device according to claim 1, wherein the sensor comprises an optical sensor including a transmitter to emit light rays and a receiver to receive the light rays.

7. The safety device according to claim 6, wherein the optical sensor comprises a light scanner that generates a binary output signal.

8. The safety device according to claim 6, wherein the housing has a cover and the transmitter and the receiver are arranged on an inside of the cover so that the light rays emitted by the transmitter are emitted in the direction of the vascular access location and from there are reflected toward the receiver.

9. The safety device according to claim 8, further including an evaluation unit coupled to the receiver and including a signal store, wherein when the cannula or a catheter are inserted correctly at the vascular access location, signals received at the receiver are stored as reference values in the signal store of the evaluation unit using a teach-in process, and during an operating phase that follows the teach-in process, actually received signals are compared to the reference values by the evaluation unit.

10. The safety device according to claim 9, further comprising an alarm coupled to the sensor to generate an alarm signal in dependence of a deviation between actually received signals and stored reference values occurring when there is a detachment of the cannula or the catheter at the vascular access location that causes leaking fluid to at least partially fill the housing so that the light rays emitted by the transmitter are reflected only by the fluid flowing in.

11. The safety device according to claim 9, wherein the sensor measures light absorption.

12. The safety device according claim 9, wherein the evaluation unit is arranged outside the housing.
